(19) United States
(12) Patent Application Publication
(10) Pub. No.: US 2005/0260163 A1
(43) Pub. Date: Nov. 24, 2005

(54) Triplex hairpin ribozyme

(76) Inventors: Joseph A. DiPaolo, Bethesda, MD (US); Luis M. Alvarez-Salas, Mexico City (MX)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/081,251

(22) Filed: Mar. 16, 2005

Related U.S. Application Data
(63) Continuation of application No. PCT/US03/29893, filed on Sep. 23, 2003.
(60) Provisional application No. 60/480,815, filed on Jun. 20, 2003.
(60) Provisional application No. 60/412,930, filed on Sep. 23, 2002.

(51) Int. Cl. A01K 67/00; A61K 48/00; C12N 5/08; A61K 9/127; C12N 15/85

(52) U.S. Cl. 424/93.2; 424/450; 435/455; 800/8

(57) ABSTRACT

A recombinant plasmid or expression vector comprising a sequence encoding a trans-acting hairpin ribozyme or inserted RNA flanked by 5′ and 3′ self-cleavage cis-acting hairpin ribozymes, which produces a long RNA transcript that undergoes self-catalyzed cleavage at the 5′ and 3′ sides of the trans-acting ribozyme or inserted RNA.

RELATED APPLICATIONS

This application is a continuation and claims the benefit of priority of International Application No. PCT/US2003/029893 filed Sep. 23, 2003, designating the United States of America and published in English on Apr. 1, 2004, as WO 2004/027044, which claims the benefit of priority of U.S. Provisional Application No. 60/480,815, filed Jun. 20, 2003, and U.S. Provisional Application No. 60/412,930, filed Sep. 23, 2002, all of which are hereby expressly incorporated by reference in their entireties.

### FIELD OF THE INVENTION

A recombinant plasmid or expression vector is provided comprising a sequence encoding a trans-acting hairpin ribozyme or inserted RNA flanked by 5′ and 3′ self-cleavage cis-acting hairpin ribozymes, which produces a long RNA transcript that undergoes self-catalyzed cleavage at the 5′ and 3′ sides of the trans-acting ribozyme or inserted RNA.

### BACKGROUND OF THE INVENTION

Experimental, epidemiological and molecular data has established that squamous cell cervical carcinomas are associated with “high” risk types of human papillomavirus (HPVs) DNA. The most important viral oncogenes found as a result of cell transformation and formation of tumors in transgenic mice with malignant types of HPVs have been E6 and E7. Their presence has been confirmed in most cervical carcinomas world-wide (Clifford, G. M. et al. 2003 Br J Cancer 88: 63-73). The continued expression of these two genes is evidence of their importance and they are often referred to as the hallmark of cervical carcinoma (Zur Hausen, H. and de Villiers, E. M. 1994 Annu Rev Microbiol 48:427-447). Furthermore, E6/E7 suppression of cervical cancer cell lines results in growth inhibition (von Knebel Doeberitz, M. et al. 1988 Cancer Res 48:3780-3786).

Human papillomaviruses are small DNA viruses that induce hyperplasia of epithelial cells. Mucosal HPVs have been well characterized and it has been reported that world-wide 20% of adult females infected are HPV positive (Koutsky, L.A. et al. 2002 N Engl J Med 347:1645-1651). Some HPVs (such as types 16 and 18) are associated with malignant progression of genital mild dysplasia to cervical cancer with type 16 (HPV-16) being the most common papillomavirus associated with cervical cancer (Zur Hausen, H. 1996 Biochim Biophys Acta 1288:F55-78). In HPV-16, both genes are expressed from a single promoter and result in polycistronic mRNA containing both E6 and E7 transcripts. E6 and E7 protein products functionally neutralize cell cycle regulatory proteins, so that cell proliferation continues.

Recently, E6 protein has been shown able to interact with a number of transcription regulators (Zimmermann, H. et al. 1999 J Virol 73:6209-6219); however, E6 primary target is the tumor suppressor p53 growth inhibitor (Gardiol, D. et al. 1999 Oncogene 18:5487-5496; Foster, S. A. et al. 1994 J Virol 68:5698-5705). Inhibition of p53 by E6 involves ubiquitin-mediated degradation of p53 and the consequent loss of p53 functions (Werness, B. A. et al. 1990 Science 248:76-79; Scheffner, M. et al. 1993 Cell 75:495-505). The E7 protein also plays an important role in the viral life cycle by subverting the tight link between cellular differentiation and proliferation in normal epithelium, thus allowing viral replication in differentiating keratinocytes that would be otherwise withdrawn from the cell cycle (Munger, K. et al. 2001 Oncogene 20:7888-7898). E7 protein from high-risk HPVs targets pRB107 and disruption of the E2F-mediated transcriptional regulation results in the up-regulation of genes required for G1/S transition and DNA synthesis (Dyson, N. et al. 1989 Science 243:934-937; Duensing, S. et al. 2001 J Virol 75:7712-7716; Munger, K. and Phelps, W. C. 1993 Biochim Biophys Acta 1155:111-123). It is the combination of E6 and E7 activities that cause genomic instability, cell immortalization and transformation leading to malignant cancer (Pirisi, L. et al. 1987 J Virol 61:1061-1066; Duensing, S. and Munger, K. 2002 Cancer Res 62:7075-7082). Additionally, E6 and E7 have been shown to induce carcinomas in transgenic mice (Herber, R. et al. 1996 J Virol 70:1873-1881; Song, S. et al. 1999 J Virol 73:5887-5893). Thus, interruption of these genes represents an ideal target for therapy.

The discovery of nucleic acids as biological catalysts (ribozymes) has been one of the most important advances in biochemistry. Progress has been obtained in understanding ribozyme reaction mechanisms, kinetics, active centers, conformational structure and minimal functional structures (Lilley, D. M. 1999 Curr Opin Struct Biol 9:330-338; Sun, L. Q., et al. 2000 Pharmacol Rev 52:325-347). The applications of small ribozymes have attracted considerable interest because of the potential methods for gene therapy through gene silencing (Birikh, K. R. et al. 1997 Eur J Biochem 245:1-16). The hammerhead and hairpin ribozymes are small cis-cleaving ribozymes found in some plant viroids and satellite RNAs and are being studied extensively. These ribozymes contain guide sequences that allow them to hybridize and subsequently cleave a specific substrate RNA. This leads to degradation of the substrate. Furthermore, because ribozymes are catalytic they may bind to other substrate molecules following cleavage of the first target. Such multiple turnover can result in more efficient inhibition (Kiehntopf, M. et al. 1994 EMBO J 13:4645-4652). Their small size and malleability make ribozymes excellent candidates as potential gene inhibitors. However, their eventual use will depend on whether they can be adapted to efficiently cleave substrates within the intracellular environment (Sullenger, B. A. 1995 Appl Biochem Biotechnol 54:57-61).

The hairpin ribozyme is a 50 nt catalytic moiety derived from the minus strand of the satellite RNA associated with tobacco ringspot virus (Haseloff, J. and Gerlach, W. L. 1988 Nature 334:585-591; Haseloff, J. and Gerlach, W. L. 1988 Nature 334:585-591). The catalytic domain of hairpin ribozymes contains two short intramolecular helices (helix 3 and helix 4) that flank and internal loop (loop B) associated with the cleavage process. Ribozyme-substrate complex is stabilized by two intermolecular helices (helix I and helix II), flanking a symmetrical internal loop (loop A) containing the substrate cleavage site. An interdomain interaction is necessary to produce catalytic activity over the target which requires minimal amounts of Mg⁺⁺ for correct positioning and no apparent dependence on co-factors for cleavage (Berzal-Herranz, A. et al. 1993 EMBO J 12:2567-2573). Cleavage occurs through a transesterification reaction pathway using the 2′-hydroxy group at the scissile linkage primary nucleophile generating cleavage products with 5′- hydroxy and 2,′,3′ cyclophosphate termini (Berzal-Herranz, A. and Burke, J. M. 1997 Methods Mol Biol 74:349-355).

A major issue in ribozyme development as therapeutic agents has been their behavior within the intracellular environment. Variables such as nuclease sensitivity, target co-localization, endogenous ion concentration and ribozyme expression levels have hampered application of ribozymes as efficient therapeutic agents (Michienzi, A. and Rossi, J. J. 2001 Methods Enzymol 341:581-596). Nevertheless, ribozymes designed to cleave targets of HIV-1, HPV, HBV and several cellular genes have been successfully tested in vitro and in vivo (Taylor, N. R. and Rossi, J. J. 1991 Antisense Res Dev 1:173-186; Alvarez-Salas, L. M. et al. 1998 PNAS USA 95:1189-1194; Feng, Y. et al. 2000 Biol Chem 382:655-660; Shore, S. K. et al. 1993 Oncogene 8:3183-3188; Irie, A. et al. 1999 Antisense Nucleic Acid Drug Dev 9:341-349). The development of nuclease-resistant ribozymes with novel nucleic acids chemistries and efficient ribozyme expression systems have greatly improved the efficiency of therapeutic ribozymes.

Our interest centers on the therapy of cervical cancer. For this purpose we also found that an antisense oligodeoxynucleotide directed to locus 434 of E6 of HPV-16 inhibited tumor cell growth and has been patented (Alvarez-Salas, L. M. et al. 1999 Antisense Nucleic Acid Drug Dev 9:441-450 and U.S. Pat. No. 6,084,090). The R434 ribozyme produces in vitro degradation of HPV-16 E6 RNA, confirming target site accessibility. It was also shown that R434 ribozyme efficiently inhibit E6/E7-mediated immortalization through the specific degradation of its mRNA when in cis-configuration (Alvarez-Salas, L. M. et al. 1998 PNAS USA 95:1189-1194).

The evolution of expression systems has led to the engineering of multiple expression (multiplex) configurations harboring several trans-acting (therapeutic) ribozymes within a single RNA molecule transcribed from RNA polymerase III promoters. A particular multiplex system uses cis-cleaving (trimming) ribozymes flanking therapeutic ribozymes allows independent action of each ribozyme thus increasing overall efficiency (Taira, K. et al. 1990 Protein Eng 3:733-737; Ohkawa, J. et al. 1993 PNAS USA 90:11302-11306). Such triplex configuration has been successfully applied to hammerhead ribozymes targeting HIV-1 and HBV (Yuyama, N. et al. 1994 Nucleic Acids Res 22:5060-5067; Ruiz, J. et al. 1997 Biotechniques 22:338-345). In contrast, no hairpin ribozyme has been expressed from any multiplex system.

### SEGUE TO THE INVENTION

Cis-cleaving hairpin ribozymes are highly efficient and can be used as trimming ribozymes to excise therapeutic ribozymes in a triplex configuration (Schmidt, C. et al. 2000 NucleicAcids Res 28:886-894). The present work focuses on the design, construction and evaluation of a triplex expression system entirely based on hairpin ribozymes. A triplex system consisting of two trimming hairpin ribozymes flanking R434 was tested for trans-cleavage with HPV-16 E6 target RNA. We demonstrate that a triplex system based on hairpin ribozymes results in high level expression of R434 leading to a more efficient cleavage of E6 mRNA than single-expressed R434 ribozymes.

### SUMMARY OF THE INVENTION

A recombinant plasmid or expression vector is provided in which DNA encoding a trans-acting hairpin ribozyme of interest is ligated to DNAs encoding other cis-acting hairpin ribozymes which serve to cleave the 5′ and 3′ ends of the trans-acting ribozyme of interest. Additionally, the trans-acting hairpin ribozyme in the recombinant plasmid or expression vector can be replaceable with any sequence (e.g., antisense RNA and RNAs of other viruses). Moreover, by connecting the whole units in tandem (shotgun-type expression system), several trans-acting hairpin ribozymes, trimmed at both 5′ and 3′ ends, are generated. By doing so, ribozymes targeted to various sites can initially be transcribed as a long RNA chain which subsequently undergoes cleavage to produce independently trans-acting ribozymes, each possessing a specific target site. The invention comprises:

(1) A recombinant plasmid or expression vector comprising a sequence encoding a trans-acting hairpin ribozyme or inserted RNA flanked by 5′ and 3′ self-cleavage cis-acting hairpin ribozymes, which produces a long RNA transcript that undergoes self-catalyzed cleavage at the 5′ and 3′ sides of the trans-acting ribozyme or inserted RNA.

(2) A recombinant plasmid or expression vector encoding 1-100 units of a trans-acting hairpin-type ribozyme flanked by 5′ and 3′ self-cleavage hairpin-type ribozymes, which produces an equivalent number of RNA transcripts connected in tandem that undergo self-catalyzed cleavage at the 5′ and 3′ sides of each trans-acting ribozyme.

(3) A method of producing the RNA transcripts self-cleaved at 5′ and 3′ sides which are transcribed from the recombinant plasmid or expression vector of (1) or (2) that act as templates.

(4) A transformant comprising a cell of a host which is transformed with the recombinant plasmid or expression vector of (1) or (2).

(5) The recombinant plasmid or expression vector of (1) or (2) wherein the trans-acting hairpin ribozyme is reduced to a self-catalytic unit by using a catalytic domain contained within the trans-acting hairpin ribozyme to cleave the trimming and therapeutic targets.

### BRIEF DESCRIPTION OF THE DRAWINGS

FIG. 1. A) Map of pTRL-5 plasmid. The relative positions of TRL, R434 and TRR ribozymes are shown within boxes. Arrow indicates the position and orientation of T3 promoter. Relevant restriction sites are marked. B) Sequence of the full TRL-R434-TRR insert is shown (SEQ ID NO: 3) and (SEQ ID NO: 4). C) Predicted secondary structure of pTRL-5 full transcript including R434, mutant tRNA^{Val} and trimming ribozymes TRL and TRR (SEQ ID NO: 1). Arrows indicate ribozyme cleavage sites. HPV-16 target sequence (nt 430-445) is shown for reference (SEQ ID NO: 7).

FIG. 2. A) Schematic representation of the triplex ribozyme processing. Full TRL-R434-TRR transcript would be cleaved into end products (circled) R434, TRL, TRR and intermediaries (boxed) TRL-R434 and R434-TRR. B) Self-processing of pTRL-5 triplex cassette. SstI-linearized (pTRL-5 SstI) and circular (pTRL-5ccc) pTRL-5 vector (one μg) were in vitro transcribed in the presence of α-[³²P]-UTP, resulting in six RNA products (1-6) separated in denaturing 6% polyacrylamide (left) or 8% polyacrylamide (right) gels. Band sizes and identities are indicated by lines. Bracketed bands correspond to incomplete transcripts.

FIG. 3. Triplex cassette self-processing. Plasmid pTRL-5 was digested with SstI or MluI endonucleases and in vitro transcribed in the presence of α-[³²P]-UTP. Processed fragments were separated in denaturing 6% polyacrylamide 7M urea gels. SstI-linearized pTRR was processed as above. Relative band sizes are indicated.

FIG. 4. Triplex cassette self-processing. Individual bands from in vitro transcribed pTRL-5 and pTRR plasmids were eluted and purified from preparative 6% polyacrylamide 7M urea gels and further incubated for 60 min in transcription buffer at 37° C. The resulting fragments from self-processing were separated in denaturing 6% polyacrylamide gels. A and B) isolated bands prior self-processing (t=0). C and D) resulting bands after 60 min incubation (t=60). Lines indicate relative mobility and size of TRL-R434-TRL, TRL-R434, R434-TRR, R434 and TRL fragments.

FIG. 5. Catalytic release of R434. A) Triplex ribozyme was produced after 30 min in vitro transcription of pTRL-5 plasmid and purified through preparative gel electrophoresis. B) Eluted TRL-R434-TRR was incubated for 0 to 60 min in transcription buffer and loaded into analytical denaturing gels. Processing was quantified by plotting the percentage of residual radioactivity relative to the total. The plot is the mean of triplicate TRL-R434-TRR (triangles) and R434 (squares) measurements, respectively. Error bars represent standard deviation.

FIG. 6. Triplex R434 cleavage of HPV-16 RNA. A) Labeled HPV-16 target RNA (nt 415 to 445) was incubated with 0.5 μg of ribozyme RNA produced from linearized templates coding for single (R434) or triplex (pTRL-5) R434 at 37° C. B) HPV-16 target RNA was incubated with 0.5 μg of ribozyme RNA produced from covalently closed circular (ccc) templates. Cleavage was calculated as the mean of the percentage of radioactivity from cleaved products relative to the input. Error bars represent standard deviation. Closed triangles, single R434; open triangles, triplex R434.

FIG. 7. Double triplex R434 activity. Cleavage kinetics of transcripts produced from covalently closed circular (ccc) templates of single (R434), double (pDR434), triplex (pTRL-5) and duplex-triplex (pDTR434) ribozymes. Cleavage was calculated as the percentage of radioactivity from cleaved products relative to total radioactivity in each lane. The plot represents the mean and standard deviation of three independent experiments. Closed triangles, 434; open triangles, pTRL-5; closed squares, pDR434; open squares, pDTR434.

### DETAILED DESCRIPTION OF THE PREFERRED EMBODIMENT

Part I

The oncoproteins of human papillomavirus type 16 (HPV-16) E6 and E7 genes efficiently immortalize cervical keratinocytes, induce tumors in transgenic mice and correlate with cervical cancer. Previously, we reported an engineered hairpin ribozyme (R434) directed against HPV-16 E6/E7 mRNA, which resulted in down-regulation of E6/E7 mRNA and inhibited growth of both HPV-16 immortalized cells and tumor cells. To improve the efficiency of R434, we now report the design and construction of a triplex expression system based entirely on hairpin ribozymes. Such system allows releasing of trans-acting (therapeutic) ribozymes from long transcripts using cis-cleaving (trimming) ribozymes. A triplex expression cassette consisting of two trimming hairpin ribozymes flanking a protected R434 was constructed. In vitro transcribed RNA from a linearized template containing the full triplex resulted in the complete release of R434 by a self-processing mechanism. Activity of trimming ribozymes was confirmed by selective templates. Triplex released R434 had better in vitro catalytic properties than that of single expressed R434. Release by trimming ribozymes allows individual activity of minimal trans-acting catalytic units resulting in increased efficiency of degradation of E6 RNA. Furthermore, duplex triplex R434 was >300% more efficient in cleaving E6 than duplex R434. Therefore, the triplex alternative is envisioned as being better suited for in vivo applications.

Part II

In order to construct a recombinant plasmid or expression vector containing the sequence encoding a trans-acting hairpin ribozyme having 5′ and 3′ self-cleavage (trimming) cis-acting hairpin ribozymes, we have combined three blocks, a 5′ block comprising the sequence encoding a 5′ cis-acting hairpin ribozyme, and the 5′ binding sequence containing its self-cleavage site, a trans-acting ribozyme block comprising the sequence encoding a trans-acting hairpin ribozyme of interest and the target binding sequence, and a 3′ block comprising the sequence encoding a 3′ cis-acting hairpin ribozyme and the 3′ binding sequence containing its self-cleavage site. The 5′ block self-cleaves the 5′ side of the trans-acting ribozyme block. The DNA sequence of the 5′ block is ligated upstream of the trans-acting ribozyme block. The 3′ block self-cleaves the 3′ side of the trans-acting ribozyme block. The 3′ block is located downstream of the trans-acting ribozyme block.

The recombinant plasmid or expression vector produces RNA transcripts in vivo (yeast, plant cell, animal cell) as well as in vitro. Transcription starts at transcription initiation site (+1) downstream of a promoter, moves down in the 3′ direction, optionally without stop even at the 3′ end of the 3′ block. The long RNA transcript undergoes self-catalyzed cleavage at the 5′ and 3′ sides of the trans-acting ribozyme block and the resulting trans-acting hairpin ribozyme has substantially minimal extra sequences at both sides.

Although the recombinant plasmid or expression vector is designed to produce the trans-acting hairpin ribozyme, the recombinant plasmid can be used to produce various RNA transcripts such as RNAs of various viruses and anti-sense RNAs simply by replacing the trans-acting ribozyme block with a sequence of interest. Various promoters can be utilized as a promoter of the recombinant plasmid or expression vector. A suitable vector can be the one capable of producing an RNA transcript in various organisms and selected according to the organisms (e.g., plant, animal).

In addition, a recombinant plasmid or expression vector can be designed to encode a number of various concatemeric units. The whole concatemeric unit (1-100 units) is placed after a promoter. A unit of the concatamer comprises a 5′ block, a trans-acting hairpin ribozyme optionally embedded into a tRNA in order to stabilize the trans-acting ribozyme (hereinafter referred to as trans-acting ribozyme/tRNA), and 3′ block. The 5′ and 3′ blocks serve to cleave the 5′ and 3′ sides of the trans-acting ribozyme/tRNA. The tRNA serves to stabilize the trans-acting ribozyme which cleaves an RNA target. The trans-acting hairpin ribozyme can be specially designed to target a specific RNA; each concatameric unit can be designed to target different RNAs. The recombinant plasmid or expression vector encoding various concatameric units targeted at different sites of the target RNA gene is therefore especially useful to target RNAs arising from microorganisms of a high mutation rate. The env gene of human immunodeficiency virus type 1 (HIV-1) and others are, for example, known to undergo mutation at a very rapid rate. The recombinant plasmid may be used to cleave the RNAs of these viruses by simultaneously targeting various sites.

A recombinant plasmid or expression vector containing the sequence encoding a trans-acting hairpin ribozyme having 5′ and 3′ self-cleavage ribozymes can produce a trans-acting ribozyme substantially free of unwanted sequence at its 5′ and 3′ flanking region without digesting the plasmid or vector with restriction enzyme. The recombinant plasmid or expression vector does not require the time-consuming digestion step as in run-off transcription. In addition, the recombinant plasmid or expression vector can produce the trans-acting hairpin ribozyme in vivo as well as in vitro. The recombinant plasmid or expression vector can be amplified in vivo while producing the trans-acting hairpin ribozyme. Additionally, efficiency of cleavage of a covalently closed circular (ccc) form is far better than that of a linearized DNA, (i.e., run-off method). This is apparently because, in the case of a covalently closed circular form, transcription occurs by a rolling circle mechanism. Furthermore, a triplex ribozyme connected in tandem was more efficient in cleavage than triplex.

As described above, the hairpin ribozyme is a 50 nt catalytic moiety derived from the minus strand of the satellite RNA associated with tobacco ringspot virus (Haseloff, J. and Gerlach, W. L. 1988 Nature 334:585-591). Ribozyme-substrate complex is stabilized by two intermolecular helices (helix I and helix II), flanking a symmetrical internal loop (loop A) containing the substrate cleavage site. The ribozyme binds to the target RNA through helix 1 (six base pairs) and helix 2 (four base pairs), separated by a NGUC loop in the substrate strand. The recognition sequence is bNGUC, where b is G, C, or U, N is any nucleotide, and cleavage occurs 5′ to the G residue. The catalytic domain of hairpin ribozymes contains two short intramolecular helices (helix 3 and helix 4) that flank an internal loop (loop B) associated with the cleavage process.

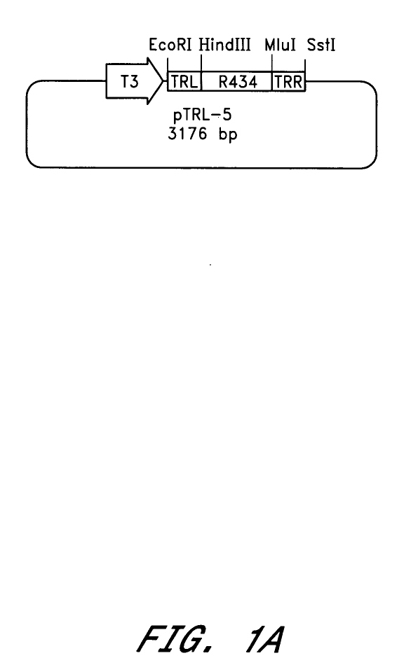

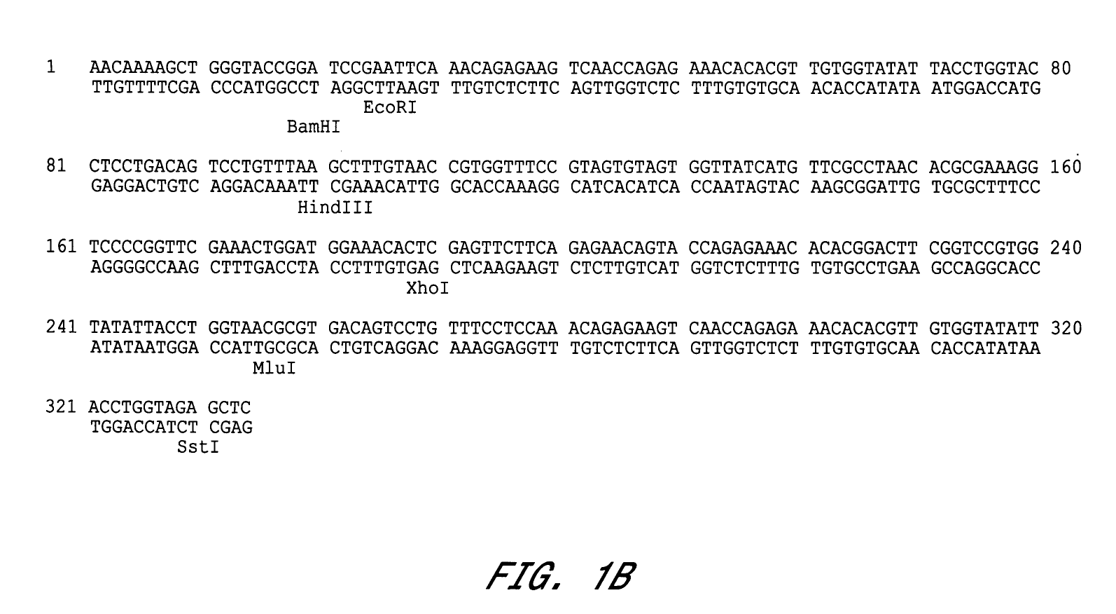

The triplex ribozymes of the present invention comprise a 5′ autocatalytically cleaving ribozyme sequence, a catalytic ribozyme comprising a target RNA-specific binding site, and a 3′ autocatalytically cleaving ribozyme. One example of the present triplex ribozyme is shown by its RNA content in FIG. 1C and SEQ ID NO: 1. The nucleotides numbered 1-329 encode the triplex ribozyme. This includes the 5′ autocatalytically cleaving ribozyme (89 b), the catalytic ribozyme protected by a tRNA (177 b), and the 3′ autocatalytically cleaving ribozyme (64 b).

The invention provides ribozymes that have the unique characteristic of being target RNA-specific in their catalytic action. In the example shown in FIG. 1C and SEQ ID NO: 1, the target RNA specificity is conferred by an RNA binding site that specifically binds a sequence that is unique to human papillomavirus type 16 (HPV-16) E6 and E7 mRNA. It will be understood that an RNA sequence unique for any RNA can be the target of the present target RNA-specific ribozyme. The determination of unique sequences is routine given the availability of numerous computer databases (GenBank) and computer programs (Genetics Computer Group, PCGENE and BLAST) which can search for and find any matches between nucleic acid sequences. A unique DNA sequence located on one of the databases will have a corresponding unique RNA sequence.

One example of the catalytic sequence of the present ribozymes is also shown as its RNA coding sequence in FIG. 1C and SEQ ID NO: 1. Other catalytic sequences include those known in the art. A number of sequence variation have defined permissible nucleotide alteration in “stem” regions. Those skilled in the art will understand that any catalytic sequence, even those not yet discovered, can be used to construct a ribozyme of the invention when it is routinely combined with the autocatalytically cleaving ribozymes and RNA binding site as described herein.

One example of the 5′ and 3′ autocatalytically cleaving ribozymes that are expressed with the catalytic ribozyme of the invention are shown in FIG. 1C and SEQ ID NO: 1. As further described below, these ribozymes are important for the expression of the catalytic ribozyme, because they cleave off of the ribozyme transcript as soon as they are transcribed to produce a catalytic ribozyme having substantially minimal extraneous 5′ or 3′ sequences. Thus, the target-specific binding site and the catalytic sequence that comprise the catalytic ribozyme are in the correct configuration to bind and cleave the target RNA. The extraneous sequences in the exemplified construct are the result of the cloning procedure. It is understood that with the selection of an alternative cloning scheme some or all of these extraneous nucleotides can be eliminated.

Ribozyme Encoding Nucleic Acids

The invention also provides nucleic acids which encode the ribozymes of the invention. These nucleic acids can be used to express the ribozymes of the invention at the selected site. The site can be tissue-specific in the case of treating tissue-specific cancers, or it can be target-specific in the case of ribozymes that prevent replication of infectious agents to treat infection (e.g. papillomavirus, hepatitis, herpes, malaria, tuberculosis, etc.).

The nucleic acids of the invention comprise a tissue-specific or non-tissue-specific promoter binding site upstream from a sequence encoding a 5′ autocatalytically cleaving ribozyme sequence, a catalytic ribozyme comprising a target RNA-specific binding site, and a 3′ autocatalytically cleaving ribozyme sequence.

The tissue-specific promoter binding site in the ribozyme-producing construct results in tissue-specific expression of the ribozyme in tissue(s) that actively transcribe RNA from the selected promoter. Thus, only the target RNA in tissue that utilizes the promoter will be cleaved by the ribozyme. The non-tissue-specific promoter results in non-tissue-specific expression and includes virus-specific promoters, such as a cytomegalovirus (CMV) promoter, and RNA polymerase III promoters.

Various tissue-specific and non-tissue-specific promoters can be used in the present nucleic acid constructs. Examples of these promoters are known to those skilled in the art. It will also be clear that target-specific promoters not yet identified can be used to target expression of the present ribozymes to the selected tissue(s) and non-tissue-specific promoters not yet identified can be used to express the present ribozymes. Once a tissue-specific promoter and non-tissue-specific promoter is identified its binding sequence can be routinely determined by routine methods such as sequence analysis. The promoter is defined by deletion analysis, mutagenesis, footprinting, gel shifts and transfection analyses.

In the ribozyme-encoding nucleic acid of the invention, the nucleic acid encoding the 5′ autocatalytically cleaving ribozyme can encode the sequence of nucleotides 1-89 shown in SEQ ID NO: 1. In the ribozyme-encoding nucleic acid of the invention, the nucleic acid encoding the 3′ autocatalytically cleaving ribozyme can encode the sequence of nucleotides 265-329 shown in SEQ ID NO: 1.

It is understood that other 5′ and 3′ autocatalytically cleaving ribozymes may be developed that can be encoded by the present nucleic acids. These ribozymes can be developed according to the methods known in the art.

The present nucleic acid encodes a catalytic ribozyme that contains two separable functional regions: a highly conserved catalytic sequence which cleaves the target RNA (also known as the “catalytic core”), and flanking regions which include a target RNA-specific binding site. By nucleic acid complementarity, the binding site directs the ribozyme core to cleave a specific site on the target RNA molecule. The length of flanking sequences have implications not only for specificity, but also for the cleavage efficiency of the individual ribozyme molecules. In the present catalytic ribozyme, the flanking sequences are highly specific for the target RNA, yet allow ready dissociation from the target RNA once cleavage occurs. This permits cycling of the ribozyme and reduces the amount of ribozyme required to be effective.

The complexity of human RNA is about 100 fold lower than that for human DNA, and specificity can be achieved with as few as 12-15 base pairs. The stability of the RNA-RNA duplex is effected by several factors, such as GC content, temperature, pH, ionic concentration, and structure. Rules known to those in the art can provide a useful estimate of the stability of the duplex.

As described above, the encoded RNA binding site is unique, so the encoding nucleic acid sequence will be the corresponding unique DNA sequence. The RNA binding site can comprise a sequence that binds to a HPV-16 E6 and E7 mRNA. The HPV-16 E6 and E7 binding site encoding RNA can have the sequence shown in FIG. 1C.

The catalytic ribozyme of the invention also includes a catalytic sequence, which cleaves the target RNA near the middle of the site to which the target RNA-specific binding site binds. In the hairpin type of ribozyme, the catalytic sequence is generally highly conserved. The conserved catalytic core residues are (SEQ ID NO: 2):

```
                    UAUAUUA A 3′
    U G C U
        GUG CUGG
U CAC GACCA 5′
    G A A
                    CAAAG
```

The most conserved and probably most efficiently cleaved sequence on the target RNA is 5′ GUC 3′. Such cleavage sites are ubiquitous in most RNAs allowing essentially all RNA's to be targeted.

With regard to the selection of the appropriate sites on target RNA, it is known that target site secondary structure can have an effect on cleavage in vitro. Thus, the selected target molecule's sequence can be routinely screened for potential secondary structure, using the program RNAFOLD (from the PCGENE group of programs or available on the Internet). Thus, reasonable predictions of target accessibility can be made. Computer assisted RNA folding, along with computational analysis for 3-dimensional modeling of RNA, is certainly effective in guiding the choice of cleavage sites.

The internal ribozyme can be targeted to noncellular RNAs necessary for growth of parasites, virus life cycles, etc., and expression can be driven with tissue-specific or non-tissue-specific promoters.

One example of the nucleic acid of the invention has the nucleotides encoding the sequence shown as SEQ ID NO: 1. This exemplary nucleic acid includes a bacterial promoter, upstream from a sequence that encodes the 5′ autocatalytically cleaving ribozyme having the sequence shown in SEQ ID NO: 1, the target binding site encoding RNA having the sequence shown in SEQ ID NO: 1, and the 3′ autocatalytically cleaving ribozyme having the sequence shown in SEQ ID NO: 1.

Alternatively, silent base substitutions in the ribozyme encoding sequence can be made that express the same ribozyme. Thus, a nucleic acid having substantially the nucleotide sequence that encodes the ribozyme shown in SEQ ID NO: 1 is provided. The nucleic acid can vary based on the characteristics/definition of the target chosen, and will have 80%-99% sequence identity with the nucleotide sequence that encodes the ribozyme shown in SEQ ID NO: 1, more preferably, it will have 90%-99% sequence identity with the nucleotide sequence that encodes the ribozyme shown in SEQ ID NO: 1. Other modifications could include for example, substitutions (or deletion or addition) of nucleotides inserted for cloning purposes and linkers. The unpaired bases can be any base, determined only by the cloning scheme chosen. If one of the bases of a pair is changed, the other must be changed in a complementary fashion. Furthermore, the ribozyme-coding sequence can be altered in ways that modify the ribozyme sequence, but do not effect the ribozyme's target RNA-specificity or negate its cleavage activity. For example, changes in the stem loop regions of the 5′, 3′, and internal ribozyme could be incorporated into other constructs while maintaining catalytic activity.

### INDUSTRIAL APPLICABILITY

Thus, this invention has several applications. The multimeric self-cleavable ribozymes of the present invention have utility for RNA-targeted gene therapy in both plants and animals to down-regulate endogenous gene expression by cleaving mRNA transcripts produced by a gene of interest. For instance, the multimeric self-cleaving ribozyme of the present invention can be used to target and cleave viral RNA in order to inhibit the replication cycle of viruses such as HIV.

The multimeric self-cleavable ribozyme of the present invention can also be used to inhibit expression of genes belonging to other infectious agents, including viruses, bacteria and protozoa, or genes whose products have deleterious effects on an organism in particular situations (e.g., inflammation in autoimmune diseases, vascular restenosis after angioplasty, defective metabolic enzymes such as the alpha-l-antitrypsin). The present invention also has application to genes involved in the control of cell growth and differentiation. These genes include those of cell cycle regulators (cyclins, cyclin dependent kinases), growth factors, growth factor receptors and second messengers, and the present invention has particular utility for the inhibition of oncogenes.

A vector comprising DNA encoding the multimeric ribozyme of the present invention can be delivered to an appropriate location in a living organism, e.g., particular organs or cell types, and the DNA incorporated in the vector can be expressed. Upon expression, the multimeric ribozyme is cleaved into its individual monomeric units, and at least one of the monomeric units recognizes and cleaves a transcript including the target recognition sequence comprising the ribozyme cleavage site transcribed from the gene of interest or a portion thereof. Thus, the transcript is cleaved and expression of the gene is down-regulated or inhibited.

The ribozyme of the present invention can also be used in virtually any application in which highly efficient, sequence-specific cleavage and destruction of RNA transcripts is desired.

Synthesis of the Ribozyme Producing Construct

Typically, the RNA binding and core sequences are synthesized as reverse complementary oligonucleotides and are cloned into a vector that will allow production of the relevant RNA containing the ribozyme. In one embodiment, the present ribozymes are prepared by synthesis of an oligonucleotide and its reverse complement. A restriction site is used in cloning. Following appropriate restriction digestion, the double-stranded DNA oligonucleotide is cloned into the cloning site within the parent vector.

Functional Testing

Once sequenced, these ribozymes are functionally tested. The test can involve transcription of the ribozyme using bacterial promoters, e.g., T3, SP6 or T7, (in the presence of trace amounts of radioactivity) followed by evaluating the autocatalytic cleavage of the ribozyme by electrophoresis. Data from these tests are provided herein.

Additional testing procedures encompass incubation of in vitro transcribed ribozymes with in vitro synthesized target RNA transcript or with cytoplasmic RNA preparations. Following incubations, RNAs are examined by standard Northern blot analyses to verify specific degradation of target RNA transcripts. Data from these tests are provided herein.

The triple-ribozyme that has been constructed can be further tested by subcloning it behind a tissue-specific promoter that will drive expression of the vector in a tissue-specific manner or behind a non-tissue-specific promoter.

The triple-ribozyme experimental approach is further validated by doing in vivo studies in mice and, ultimately, in humans.

Delivery

The nucleic acids of the invention can be in a vector for delivering the nucleic acid to the site for expression of the ribozyme. The vector can be one of the commercially available preparations. Vector delivery can be by liposome, using commercially available liposome preparations or newly developed liposomes having the features of the present liposomes. Other delivery methods can be adopted and routinely tested in methods known to those skilled in the art.

The modes of administration of the liposome will vary predictably according to the disease being treated and the tissue being targeted. For lung (e.g., tuberculosis, cancer) and liver (e.g., hepatitis and cancer) which are both sinks for liposomes, intravenous administration is reasonable. For many other localized pathologic conditions including cancers, infections (e.g., hepatitis, cystitis, proctitis, cervicitis, etc.) as well as precancerous conditions, catheterization of an artery upstream from the organ is a preferred mode of delivery, because it avoids significant clearance of the liposome by the lung and liver. For lesions at a number of other sites (e.g., skin cancer, human papilloma virus infection, herpes (oral or genital) and precancerous cervical dysplasia), topical delivery is expected to be effective and may be preferred, because of its convenience.

Leukemias and other conditions such as malaria, may also be more readily treated by ex vivo administration of the ribozyme.

The liposomes may be administered topically, parenterally (e.g., intravenously), by intramuscular injection, by intraperitoneal injection, transdermally, excorporeally or the like, although IV or topical administration is typically preferred. The exact amount of the liposomes required will vary from subject to subject, depending on the species, age, weight and general condition of the subject, the severity of the disease that is being treated, the particular compound used, its mode of administration, and the like. Thus, it is not possible to specify an exact amount. However, an appropriate amount may be determined by one of ordinary skill in the art using only routine experimentation given the teachings herein. Generally, dosage will approximate that which is typical given in antisense methodology

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained.

Topical administration can be by creams, gels, suppositories and the like. Ex vivo (excorporeal) delivery can be as typically used in other contexts.

Further, the effect of the present invention can also be obtained by incorporating the DNA of the present invention into a suitable viral gene vector and administering said vector into the body to express the ribozyme polyribonucleotide in cells. Recombinant retrovirus and vaccinia virus are examples of such vectors.

Transgenic Animals

The invention provides a transgenic non-human animal, containing, in a germ or somatic cell, a nucleic acid comprising a target-specific or RNA polymerase III promoter binding site upstream from a sequence encoding a 5′ autocatalytically cleaving ribozyme sequence, a catalytic ribozyme comprising a target RNA-specific binding site, and a 3′ autocatalytically cleaving ribozyme sequence, wherein the animal expresses a ribozyme comprising a 5′ autocatalytically cleaving ribozyme sequence, a catalytic ribozyme comprising a target RNA-specific binding site, and a 3′ autocatalytically cleaving ribozyme sequence.

The nucleic acid can be the nucleic acid shown in the figures. Alternatively, silent base substitutions in the ribozyme encoding sequence can be made that express the same ribozyme. For example, these substitutions can be as described above.

The transgenic non-human animal of the invention is useful, because the animal does not express a phenotype associated with the target RNA (e.g., with the protein it encodes). As used herein the term “phenotype” includes morphology, biochemical profiles (e.g., changes in amounts of RNA or protein expressed, etc.) and other parameters that are affected by the knockout. For example, cell death of otherwise healthy cells can be a measure of altered phenotype resulting from ribozyme expression.

Transformed Host Cells

The present ribozymes can be expressed in a transformed cell line. The transformed cell can be used to validate both the specificity of the ribozyme's expression and the specificity and cleavage activity against the target RNA. Examples of such a screening function are known in the art.

Screening Methods

The transgenic animals and transformed host cells of the invention can be used in a method of screening a compound for its ability to cause the animal or host cell to express a phenotype associated with the target RNA. The method requires administering the compound to the animal/cell and assessing the compounds ability to cause expression of the phenotype. If the phenotype is restored, the compound is considered to be effective. For example an L-dopa functional knockout transgenic animal can be made and used to screen for drugs that restore an L-dopa associated phenotype.

Treating Proliferative Diseases

A method of treating a subject having a proliferative disease is provided. The treatment is carried out by inhibiting cell proliferation, and this is accomplished by administering to the subject a nucleic acid encoding a ribozyme that is targeted to an RNA that is essential to cell growth. The ribozyme encoded by the nucleic acid is expressed, production of an essential RNA is inhibited, cell proliferation is inhibited, cell death ensues and the proliferative disease treated. For example, the invention provides a method of treating a subject having cervical cancer comprising administering to the subject the nucleic acid encoding SEQ ID NO: 1, whereby the ribozyme encoded by the nucleic acid is expressed in the cervix and the cervical cancer is treated.

Treating Viral Infection

A method is provided of treating a viral infection in a subject, comprising administering to the subject a nucleic acid of the invention, wherein the encoded target RNA-specific binding site is specific for an RNA unique to the infectious agent, whereby the ribozyme encoded by the nucleic acid is expressed and the infectious agent is killed. Transcription can be driven using a non-tissue-specific promoter or a tissue-specific promoter which will selectively express the targeted ribozyme in virus-infected tissue, i.e., using the liver-specific albumin promoter for expression of a targeted ribozyme directed against hepatitis B virus.

In the context of determining anti-viral efficacy, ribozyme expressing cell lines can be compared with their ribozyme negative counterparts for their ability to support viral infection/replication/yield. In a manner similar to that described above, ribozyme expressing cell lines can be obtained and assayed; and in all cases the abilities of the ribozyme to prevent infection can be determined.

Part III

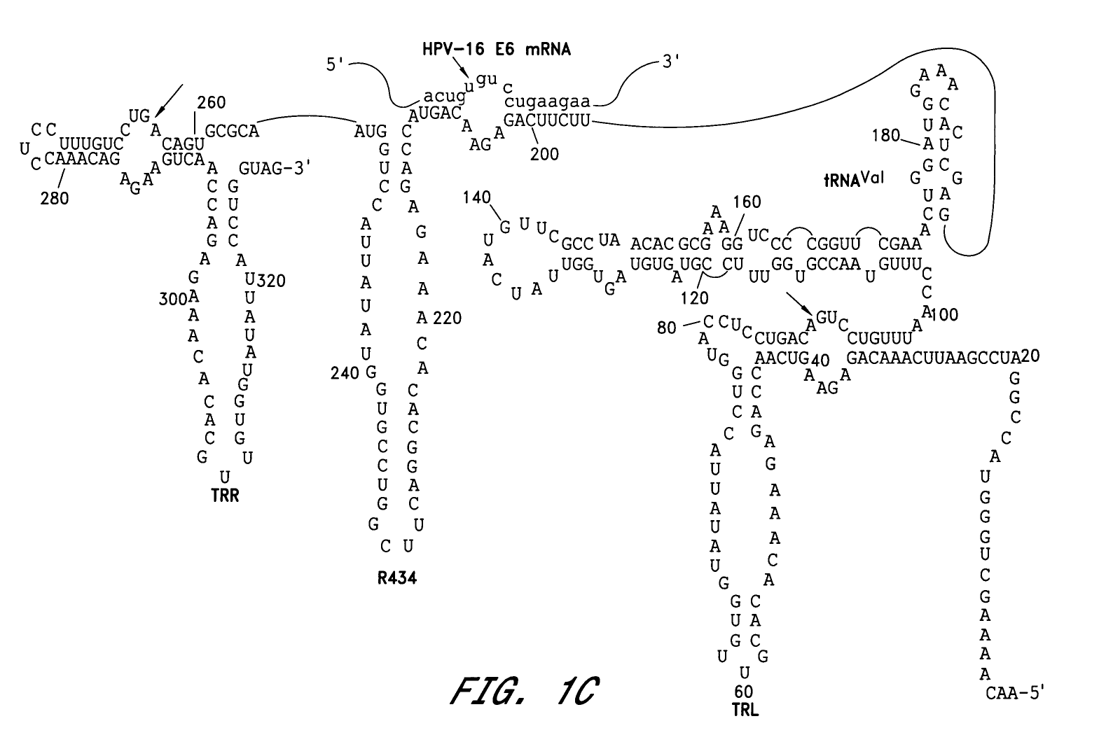

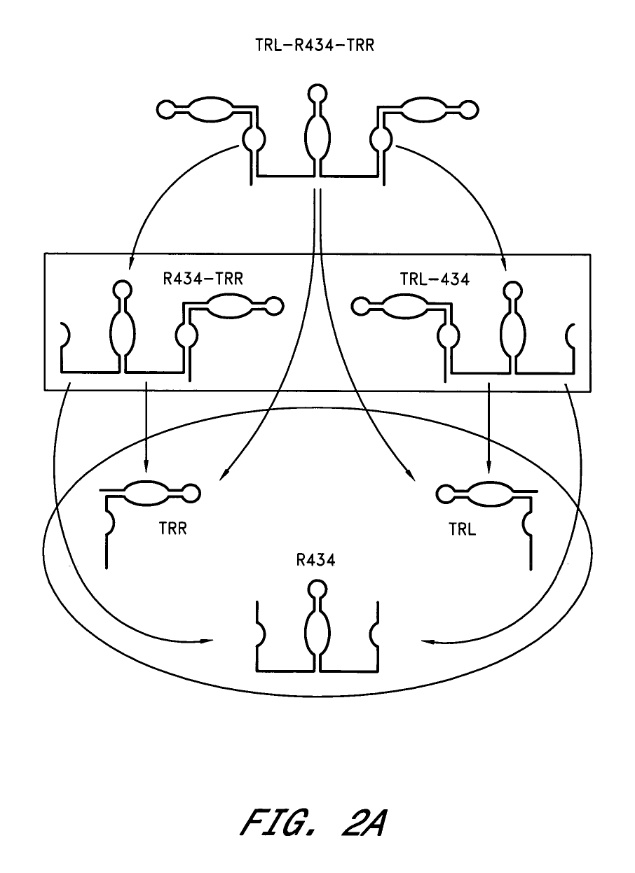

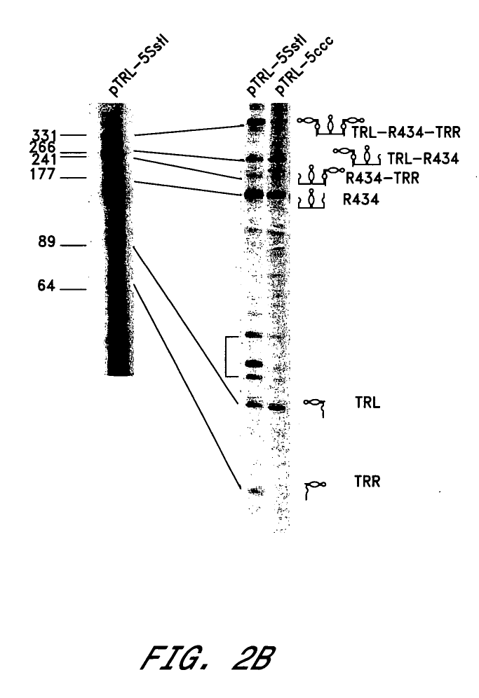

Self-processing of the triplex ribozyme system. The triplex hairpin ribozyme system consisted of two trimming hairpin ribozymes (TRL and TRR) flanking the R434 therapeutic ribozyme, cloned in a pBS KS-vector. The resulting plasmid pTRL-5 contains the triplex cassette under control of the T3 promoter (FIG. 1A and 1B). In this configuration, TRL target strand (containing the scissile 5′-GUC-3′) should turn 180° at the 3′ end of the domain B in order to release R434 (FIG. 1C). In the native configuration, the target RNA strand turns itself on the 5′ end for cis-cleavage, as shown for TRR (Berzal-Herranz, A. et al. 1993 EMBO J 12:2567-2573). Thus, self-cleavage process of the triplex TRL-R434-TRR (329 b) would yield three end products including R434 5′ protected by a mutant tRNA^{Val} (177 b) and trimming ribozymes TRL and TRR (89 and 64 b, respectively). Intermediary products TRL-R434 and R434-TRR (266 and 241 b, respectively) may be also present as a part of the self-cleavage process (FIG. 2A).

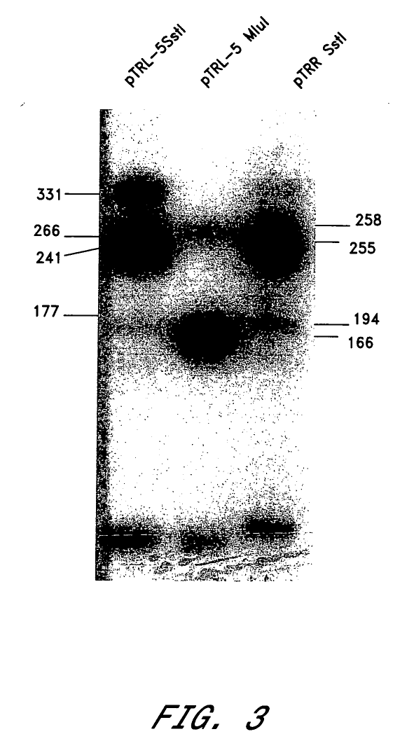

Initial experiments showed that in vitro transcribed pTRL-5 yielded the six products of the expected size when linearized with SstI restrictase, which allows for transcription of the full triplex cassette. The six fragments are consistent with the presence of mixed end and intermediary self-cleavage products (FIG. 2B). Transcription of covalently closed circled (ccc) pTRL-5 template resulted in the expected fragments lacking TRR (FIG. 2B). In vitro transcription with MluI-digested pTRL-5 template resulted in the expected three fragments corresponding in size to TRL-R434 (255 b), protected R434 (166 b) and TRL (89 b) products. Digestion with MluI impedes transcription of TRR and consequently there was no presence of TRL-R434-TRR full product or R434-TRR intermediary (FIG. 3). These results confirmed that correct processing by TRL trimming ribozyme had occurred.

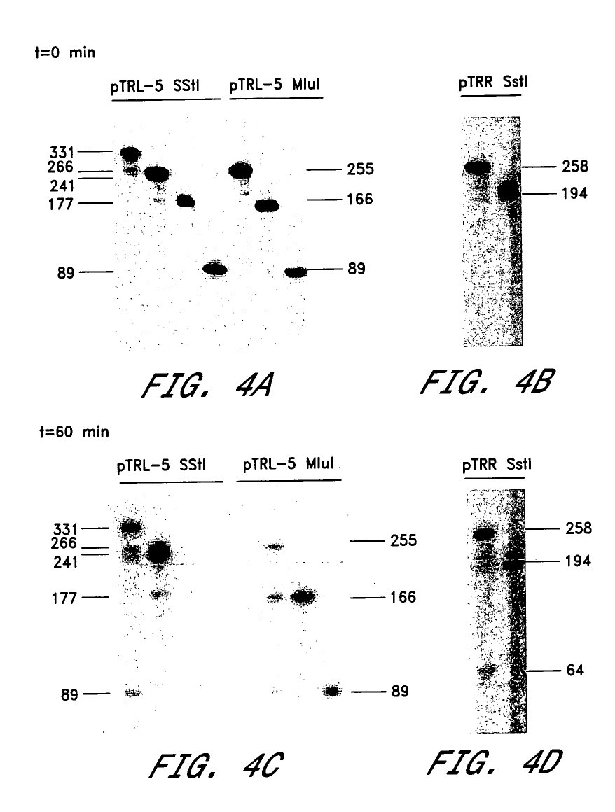

Further evidence of self-processing was obtained through elution of individual RNA fragments followed by one hour incubation at 37° C. Here, each pTRL-5 transcript self-processed and the expected products were produced: fragment 1 in TRL-R434, R434-TRR, R434 and the trimming ribozymes; fragments 2 and 3 in TRL, R434 and TRR. Fragments 4, 5 and 6 had no self-processing because they are products (FIG. 4A-4D). Therefore, TRL and TRR trimming ribozymes efficiently release R434 ribozyme.

TRR activity was tested using the pTRR plasmid, a derivative of pTRL-5 lacking EcoRI-HindIII fragment and therefore contain only the R434 and TRR ribozymes. The pTRR transcription and self-cleavage resulted in three products corresponding to the R434-TRR (258 b) and the R434 (194 b) and TRR (64 b) end products (FIG. 4A-4D). Difference in size between pTRL-5 and pTRR products was due to the pTRR construct, which added 17 b to R434. Transcription of pTRL-5 template incorporating fluorescein-12-uridine-5′-triphosphate inhibited hairpin ribozyme activity and thus only the full TRL-R434-TRR transcript was produced, further confirming the need of trimming ribozymes for R434 release.

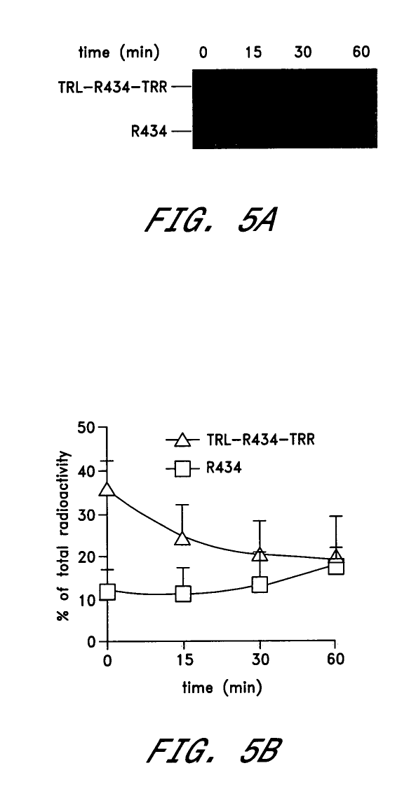

After 15, 30 and 60 min transcription reactions R434 release was measured by counting residual radioactivity of the 177 b band relative to the full-transcript 329 b band. The increase in R434 plateau at 15 min was accompanied by a decrease on TRL-R434-TRR, again indicating processing of the full transcript (FIG. 5A and 5B). These results clearly indicate that R434 is efficiently released from the full-length transcript by the activity of trimming ribozymes.

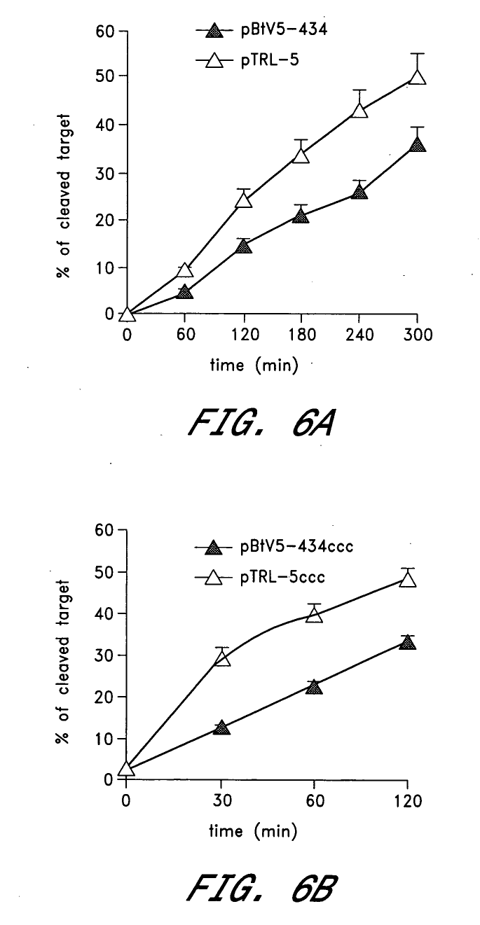

Trans-cleavage activity of triplex expressed R434. The effect of trimming ribozymes on R434 activity was tested by incubating equimolar amounts of triplex and single R434 RNA produced from linear templates with a radiolabeled transcript containing the HPV-16 R434 target site (nt 410 to 445). After one hour, triplex-expressed R434 was marginally more efficient than its single counterpart. However, efficiency of triplex R434 (pTRL-5) increased to 20% over single R434 after four hours incubation (FIG. 6A) indicating that participation of trimming ribozymes may enhance R434 activity.

After two hours incubation expression using covalently closed circular (ccc) templates resulted in that triplex R434 was 30% more efficient than single R434 in cleaving the target sequence (FIG. 6B). Because each experiment produced similar amounts of R434, these differences do not reflect changes in R434 activity itself, but indicate the participation of TRL and TRR in the release of R434 from the long transcripts produced in circular templates thus enhancing overall trans-cleavage activity.

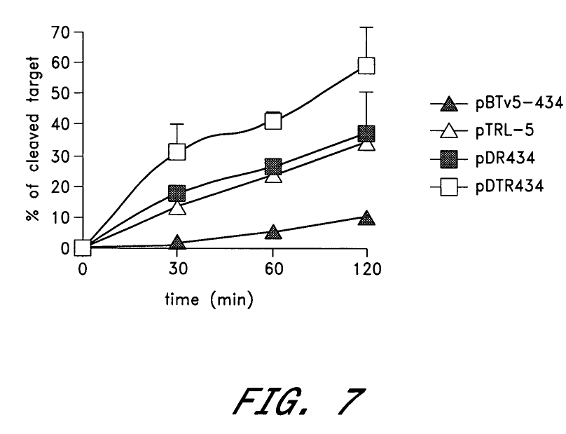

A duplex-triplex R434 construct containing tandem copies of the TRL-R434-TRR cassette was constructed (pDTR434) and transcribed to compare activity to triplex and single R434 ribozymes (FIG. 7). As ribozyme copy number control a simple duplex ribozyme was constructed (pDR434). Interestingly, cleavage activity of the duplex-triplex R434 (DTR434) RNA expressed from circular templates resulted in over 300% more target cleavage than R434. Moreover, DTR434 cleavage activity was twice that of triplex R434 (FIG. 7). This is due to the activity of the individual R434 units released by trimming the ribozymes. Therefore, a multiple triplex hairpin system can be adapted to express several ribozymes against the same or different targets to result in increased overall cleavage activity.

Part IV

The ability to design ribozymes to cleave target RNAs catalytically in trans has led to their study as gene inhibitors in vivo. The substrate requirements for both hammerhead and hairpin ribozymes can be readily determined. Therefore, because of their small size, they are excellent candidates for gene therapy and viral inhibition. The concept of using various antisense technologies complementary to a specific target is an area that we have been investigating as an approach for the treatment of cervical cancer (Alvarez-Salas, L. M. et al. 1998 PNAS USA 95:1189-1194; Alvarez-Salas, L. M. et al. 1999 Antisense Nucleic Acid Drug Dev 9:441-450). We have focused on hairpin ribozymes and have shown that that they can form a complex with their complementary target, HPV-16 E6/E7 RNA, resulting in cleavage and degradation of the target. The target is positioned so that, because of its polycistronic nature, the RNA E7 is inhibited in addition to E6. Our primary focus has been R434 because of its superior catalytic efficiency against HPV-16 E6/E7 RNA. E6 translation was inhibited in vitro and cis-expression of the ribozyme prevented human keratinocytes immortalization by HPV-16 E6/E7 (Alvarez-Salas, L. M. et al. 1998 PNAS USA 95:1189-1194).

Hairpin ribozyme cleavage relies on the target complementarity of nucleosides present in the catalytic domain and the tertiary structure of the ribozyme (Esteban, J. A. et al. 1997 J Biol Chem 272:13629-13639; Walter, N. G. et al. 1998 EMBO J 17:2378-2391). There is minimal need of divalent cations for ribozyme folding and docking (Chowrira, B. M. et al. 1993 Biochemistry 32:1088-1095). For therapeutic purposes hairpin ribozymes require a stable and predictable behavior in vivo. However, a major obstacle for hairpin ribozyme therapeutics is their relatively larger size that limits efficient chemical synthesis. Currently, hairpin ribozyme application is best suited for viral delivery. Although powerful promoters have been used to improve ribozyme expression, in vivo performance of hairpin ribozymes is compromised because of the limited amount of ribozyme produced and therefore novel expression systems required. The ability to successfully construct a series of triplex models containing ribozymes against several targets makes it possible to increase gene inhibition using standard delivery systems.

Hammerhead ribozymes have been first tested as trimming moieties for triplex systems because their catalytic domain is centered between two hybridization domains, thus avoiding complex structural manipulations to properly align the target. Multiple triplex expression systems based on hairpin ribozymes increased overall cleavage efficiency of HIV-1 and retinoblastoma gene mRNA (Ohkawa, J. et al. 1993 PNAS USA 90:11302-11306; Benedict, C. M. et al. 1998 Carcinogenesis 19:1223-1230). In 1994 Daros et al., studying replication of avocado sunblotch tobacco viroid described a synthetic pathway with two rolling circles and hammerhead ribozyme processing (Daros, J. A. et al. 1994 PNAS USA 91:12813-12817). However, such systems have not been applied to hairpin ribozymes. This is due to the structure of the hairpin ribozyme itself. Hairpin ribozymes hybridize and cleave a target sequence located 5′ of the catalytic domain B. This architecture complicates a triplex design because one of the trimming ribozymes require either a loop 3′ of the catalytic domain to position the target sequence, or the target sequence itself must be located 3′ of the catalytic domain. The latter approach has been successfully tested on duplex hairpin ribozymes using reverse-joined domains, but not on a triplex design (Schmidt, C. et al. 2000 Nucleic Acids Res 28:886-894).

The present work describes an effective triplex system based fully on hairpin ribozymes directed against HPV-16 E6/E7 mRNA. Ribozyme R434 was used as a therapeutic moiety flanked by two trimming ribozymes. The triplex system was designed using a 3′ loop on one trimming ribozyme (TRL), which successfully self-cleaved allowing R434 release. The other trimming ribozyme (TRR) is a standard hairpin self-cleaving moiety and, as expected, efficiently cleaved its target sequence. Functionality of the triplex system relied upon the success in cleaving TRL target site at 180° degrees from its native position. Simultaneous activity of both trimming ribozymes resulted in efficient release of R434 with retention of its catalytic properties, as shown in FIG. 2B using linearized and covalently closed circular (ccc) templates with the expected products being formed.

Furthermore, triplex release of R434 from long transcripts allowed superior HPV-16 target cleavage from circular templates compared to the single expressed R434. A duplex triplex construct (pDTR434) containing two tandem copies of the triplex cassette was even more efficient (>300%) in cleaving HPV-16 RNA, indicating the use of our design for multiple ribozyme expression. The synergistic activity obtained with the triplex indicates the use of cassettes containing different ribozymes that would be more efficient than their non-triplex counterparts.

The triplex system is contemplated as being reduced to a much smaller self-catalytic unit by using the catalytic domain (domain B) of R434 to cleave the trimming and therapeutic targets. Such configuration has been reported to be functional (Komatsu, Y. et al. 1997 Biochemistry 36:9935-9940), due to the capacity of hairpin ribozymes to form catalytic four-way junctions with isolated B domains (Shin, C. et al. 1996 Nucleic Acids Res 24:2685-2689; Walter, F. et al. 1998 Biochemistry 37:17629-17636). In contrast, a conventional hammerhead-based triplex cannot be modified due to the structural characteristics of hammerhead ribozymes. R434 has been shown as an in vivo inhibitor of HPV-16 E6/E7 expression. Therefore, implementation of a triplex system that significantly enhanced R434 activity is envisioned as an alternative to the antisense treatment of cervical cancer.

### EXAMPLE 1

Oligodeoxynucleotides and plasmids. Plasmid pTRR was made by inserting the double stranded oligodeoxynucleotide (dsODN) 5′-CGC GTG ACA GTC CTG TTT CCT CCA AAC AGA GAA GTC AAC CAG AGA AAC ACA CGT TGT GGT ATA TTA CCT GGT AGA GCT-3′ (SEQ ID NO: 5) into the MluI/SstI sites of pBtV5-434 plasmid containing the R434 ribozyme flanked by a mutated tRNA^{Val} and a tetraloop (Alvarez-Salas, L. M. et al. 1998 PNAS USA 95:1189-1194). Triplex expression plasmid pTRL-5 was constructed by cloning the dsODN 5′-AAT TCA AAC AGA GAA GTC AAC CAG AGA AAC ACA CGT TGT GGT ATA TTA CCT GGT ACC TCC TGA CAG TCC TGT TTA-3′ (SEQ ID NO: 6) into the EcoRI/HindIII sites of pTRR (FIGS. 1A and 1B). The duplex triplex construct pDTR434 with two copies of the triplex cassette was made by cloning tandem copies of PCR-amplified EcoRI-SstI fragment from pTRL-5. The pDR434 plasmid contains tandem copies of R434 ribozyme on the pBS KS⁻ vector (Stratagene, La Jolla Calif.). All plasmids were manually sequenced prior in vitro transcription experiments using Sequenase V.2.0 (Amersham Biosciences, Piscataway N.J.).

In vitro transcription. Plasmid minipreps from pBtV5-434, pTRL-5 and pTRR were linearized with either SstI or MluI restriction endonucleases and purified by phenol-chloroform-isoamyl alcohol (25:24:1) extraction. One μg of linearized plasmid DNA was incubated with the T3 RiboProbe in vitro transcription system (Promega Inc., Madison Wis.) in the presence of α-[³²P]-UTP (3000 Ci/mmol, Amersham Pharmacia Biotechnologies Inc.), as described by the manufacturer. Labeled transcripts were loaded into 8% polyacrylamide 7M urea gels and electrophoresed at 250V. Dried gels were exposed to X-OMAT radiographic films (Kodak Inc., N.J.). Alternatively, fragments were excised from the gels and eluted in 350 μl of E buffer (1 mM EDTA, 0.5M ammonium acetate, 0.1% SDS, 20 U RNaseA inhibitor) overnight at 4° C. For fluorescence labeling, radiolabeled UTP was substituted by fluorescein-12-uridine-5′-triphosphate (Roche Diagnostics GmbH, Mannheim) in the labeling reaction.

Ribozyme cleavage assays. Ribozyme RNA was obtained by in vitro transcription using 0.25 μM UTP and incubation with a radiolabeled target RNA containing HPV-16 nt 410-445 in RZ buffer (10 mM Tris-HCl pH 7.0, 2 mM MgCl₂, spermidine) at 37° C., as previously described (Alvarez-Salas, L. M. et al. 1998 PNAS USA 95:1189-1194). Cleavage products were separated through denaturing polyacrylamide gel electrophoresis. For circular templates, one μg of plasmid DNA was incubated directly with the T3 RiboProbe system for 30 min before addition of labeled target RNA. Dried gels were exposed to X-OMAT films and quantified in a Typhoon 8600 fluorographic scanner (Amersham BioSciences). Self-cleavage was measured by plotting the percentage of residual radioactivity from individual fragments relative to input radioactivity. Cleavage activity was then plotted as the percentage of radioactivity from processed bands relative to total radioactivity.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A recombinant plasmid or expression vector comprising a sequence encoding a trans-acting hairpin ribozyme or inserted RNA flanked by 5′ and 3′ self-cleavage cis-acting hairpin ribozymes, which produces a long RNA transcript that undergoes self-catalyzed cleavage at the 5′ and 3′ sides of the trans-acting ribozyme or inserted RNA.

2. A recombinant plasmid or expression vector encoding 1-100 units of a trans-acting hairpin-type ribozyme flanked by 5′ and 3′ self-cleavage hairpin-type ribozymes, which produces an equivalent number of RNA transcripts connected in tandem that undergo self-catalyzed cleavage at the 5′ and 3′ sides of each trans-acting ribozyme.

3. A method of producing RNA transcripts self-cleaved at 5′ and 3′ sides comprising subjecting the recombinant plasmid or expression vector of claims 1 or 2 to transcription conditions and allowing RNA transcripts to be self-cleaved.

4. A transformant comprising a cell of a host which is transformed with the recombinant plasmid or expression vector of claims 1 or 2.

5. The recombinant plasmid or expression vector of claims 1 or 2 wherein the trans-acting hairpin ribozyme is reduced to a self-catalytic unit by using a catalytic domain contained within the trans-acting hairpin ribozyme to cleave the trimming and therapeutic targets.

6. The recombinant plasmid or expression vector of claims 1 or 2 further comprising a tissue-specific or virus-specific promoter binding site upstream from said sequence encoding said trans-acting hairpin ribozyme or inserted RNA flanked by 5′ and 3′ self-cleavage cis-acting hairpin ribozymes.

7. The recombinant plasmid or expression vector of claims 1 or 2 comprising a sequence that encodes the 5′ autocatalytically cleaving ribozyme having the sequence shown in SEQ ID NO: 1, the target binding site encoding RNA having the sequence shown in SEQ ID NO: 1, and the 3′ autocatalytically cleaving ribozyme having the sequence shown in SEQ ID NO: 1.

8. The recombinant plasmid or expression vector of claims 1 or 2 comprising a sequence that encodes the 5′ autocatalytically cleaving ribozyme having the sequence shown in SEQ ID NO: 1, the target binding site encoding RNA having the sequence shown in SEQ ID NO: 1, or the 3′ autocatalytically cleaving ribozyme having the sequence shown in SEQ ID NO: 1.

9. The recombinant plasmid or expression vector of claims 1 or 2 comprising the nucleotide sequence that encodes the ribozyme shown in SEQ ID NO: 1.

10. The recombinant plasmid or expression vector of claims 1 or 2 comprising a sequence having 80%-99% sequence identify with the nucleotide sequence that encodes the ribozyme shown in SEQ ID NO: 1.

11. The recombinant plasmid or expression vector of claims 1 or 2 comprising a sequence having 90%-99% sequence identity with the nucleotide sequence that encodes the ribozyme shown in SEQ ID NO: 1.

12. The recombinant plasmid or expression vector of claims 1 or 2, wherein the catalytic domain of the trans-acting hairpin ribozyme, 5′ self-cleavage cis-acting hairpin ribozyme, or 3′ self-cleavage cis-acting hairpin ribozyme has the sequence of SEQ ID NO: 2.

13. The recombinant plasmid or expression vector of claims 1 or 2 encoding SEQ ID NO: 1, wherein the target binding site binds to a different RNA sequence.

14. The transcript encoded by the recombinant plasmid or expression vector of claims 1 or 2.

15. A liposome preparation comprising the recombinant plasmid or expression vector of claims 1 or 2 in combination with a liposome.

16. A recombinant virus comprising the sequence of the recombinant plasmid or expression vector of claims 1 or 2 in combination with a viral gene vector.

17. A method of treating a subject having a proliferative disease by inhibiting cell proliferation comprising administering to the subject the recombinant plasmid or expression vector of claims 1 or 2 in which the trans-acting hairpin ribozyme is targeted to an RNA that is essential to cell growth, whereby the transcript is expressed, production of an essential RNA is inhibited, cell proliferation is inhibited, and the proliferative disease is treated.

18. A method of treating a subject having cervical cancer comprising administering to the subject the recombinant plasmid or expression vector of claims 1 or 2 encoding SEQ ID NO: 1, whereby the transcript is expressed in the cervix and the cervical cancer is treated.

19. A method of treating a viral infection in a subject comprising administering to the subject the recombinant plasmid or expression vector of claims 1 or 2, wherein the encoded target RNA-specific binding site is specific for an RNA unique to the infectious agent, whereby the transcript is expressed and the infectious agent is killed.

20. A transgenic non-human animal, containing, in a germ or somatic cell, the recombinant plasmid or expression vector of claims 1 or 2.


SEQUENCE LISTING (7 sequences)

SEQ ID NO: 1 - RNA, 329 nt, Artificial Sequence
  triplex ribozyme
aacaaaagcuggguaccggauccgaauucaaacagagaagucaaccagagaaacacacguugugguauauuaccugguaccuccugacaguccuguuuaagcuuuguaaccgugguuuccguaguguagugguuaucauguucgccuaacacgcgaaagguccccgguucgaaacuggauggaaacacucgaguucuucagagaacaguaccagagaaacacacggacuucgguccgugguauauuaccugguaacgcgugacaguccuguuuccuccaaacagagaagucaaccagagaaacacacguugugguauauuaccugguag

SEQ ID NO: 2 - RNA, 36 nt, Artificial Sequence
  synthetic catalytic core of a triplex ribozyme
accagagaaacacacguugugguauauuaccuggua

SEQ ID NO: 3 - DNA, 334 bp, Artificial Sequence
  a strand of the plasmid insert
aacaaaagctgggtaccggatccgaattcaaacagagaagtcaaccagagaaacacacgttgtggtatattacctggtacctcctgacagtcctgtttaagctttgtaaccgtggtttccgtagtgtagtggttatcatgttcgcctaacacgcgaaaggtccccggttcgaaactggatggaaacactcgagttcttcagagaacagtaccagagaaacacacggacttcggtccgtggtatattacctggtaacgcgtgacagtcctgtttcctccaaacagagaagtcaaccagagaaacacacgttgtggtatattacctggtagagctc

SEQ ID NO: 4 - DNA, 334 bp, Artificial Sequence
  complementary strand of the plasmid insert
ttgttttcgacccatggcctaggcttaagtttgtctcttcagttggtctctttgtgtgcaacaccatataatggaccatggaggactgtcaggacaaattcgaaacattggcaccaaaggcatcacatcaccaatagtacaagcggattgtgcgctttccaggggccaagctttgacctacctttgtgagctcaagaagtctcttgtcatggtctctttgtgtgcctgaagccaggcaccatataatggaccattgcgcactgtcaggacaaaggaggtttgtctcttcagttggtctctttgtgtgcaacaccatataatggaccatctcgag

SEQ ID NO: 5 - DNA, 78 bp, Artificial Sequence
  oligodeoxynucleotide
cgcgtgacagtcctgtttcctccaaacagagaagtcaaccagagaaacacacgttgtggtatattacctggtagagct

SEQ ID NO: 6 - DNA, 75 bp, Artificial Sequence
  oligodeoxynucleotide
aattcaaacagagaagtcaaccagagaaacacacgttgtggtatattacctggtacctcctgacagtcctgttta

SEQ ID NO: 7 - RNA, 16 nt, HPV-16
acuguguccugaagaa